(19) United States
(12) Patent Application Publication
(10) Pub. No.: US 2021/0169696 A1
(43) Pub. Date: Jun. 10, 2021

(54) MEDICAL DEVICE FOR GLAUCOMA SURGERY WITH CONTROLLED AND MODULABLE FILTRATION

(71) Applicants: Paolo CECCHINI, Trieste (IT); Luigino Zovatto, Loiri Porto San Paolo (Sassari) (IT)

(72) Inventors: Paolo CECCHINI, Trieste (IT); Luigino Zovatto, Loiri Porto San Paolo (Sassari) (IT)

(21) Appl. No.: 17/110,480

(22) Filed: Dec. 3, 2020

(30) Foreign Application Priority Data
Dec. 5, 2019 (IT) 102019000023106

(51) Int. Cl. A61F 9/007; A61F 9/008

(52) U.S. Cl. CPC A61F 9/00781; A61F 2009/00891; A61F 2009/00865; A61F 9/008

(57) ABSTRACT

A medical device for glaucoma surgery includes a tubular body provided with a through cavity extending from a proximal end to a distal end. The tubular body has a first proximal positioning means at the proximal end, and a second distal positioning means at the distal end. The tubular body has at least one proximal filtration hole, located on the side of the proximal end, and at least one distal filtration hole, located on the side of the distal end, the proximal and distal holes being fluidically connected to each other by the through cavity. The proximal and distal filtration holes are at least partially or completely occluded by a membrane made of resorbable material at least partially removable by selective action, so as to allow selective variation of the amount of aqueous humor flow removable via the through cavity.

### CROSS-REFERENCE TO RELATED APPLICATION

This application claims priority to Italian Patent Application No. 102019000023106 filed on Dec. 5, 2019, the entire contents of which is hereby incorporated in its entirety by reference

### FIELD OF THE INVENTION

The present invention relates to an improved medical device for open-angle glaucoma surgery with controlled and modular filtration.

### BACKGROUND OF THE INVENTION

As is known, glaucoma is an ocular disease characterized by progressive loss of retinal ganglion cells and their axons. The functional consequence is the progressive appearance and subsequent deepening of scotomatous areas (i.e. areas of reduced light sensitivity) to the visual field up to a complete loss of the visual function. There are numerous types of glaucoma. The most common is chronic open-angle glaucoma. This form of glaucoma is characterized by high ocular pressure, a wide irido-sclero-corneal junction and damage to the nerve fibers that make up the optic nerve.

The exact cause is not yet fully understood. However, there is evidence of an involvement of some genes. The pathogenetic mechanism involves a spongiform structure called trabeculate, located at the level of the irido-sclero-corneal junction, responsible for allowing the aqueous humor to flow towards Schlemm's canal and from this into the venous stream. In primary open-angle glaucoma, an impediment at the level of the trabeculae, by preventing a correct drainage of the aqueous humor, would induce an increase in endo-ocular pressure. The hypertonicity may cause the death by apoptosis of the ganglion cells that make up the optic nerve. Typically, a thinning of the so-called neural rim of the optic nerve occurs, which appears pale and excavated, with progressive and irreversible damage to the visual field. Similar, but often more aggressive, forms of glaucoma are pseudoexfoliative glaucoma and pigmentary glaucoma. Pseudoexfoliative glaucoma is characterized by the presence of whitish furfural material, while pigmentary glaucoma is characterized by the presence of pigment granules. Both phenomena may induce an increase in intraocular pressure since the exfoliated or dispersed material is deposited in the irido-sclero-corneal junction at the level of the trabeculae, preventing the correct flow of the aqueous humor.

A first approach to treatment of glaucoma consists in instillation of hypotonizing eye drops. Some molecules reduce the production of aqueous humor, while others promote the outflow thereof. Another therapeutic possibility consists in trabeculoplasty laser, that is a laser treatment at the trabecular level, which aims to increase the outflow of aqueous humor.

When medical therapy based on hypotonizing eye drops is not sufficient to control the progression of glaucomatous damage, a surgical approach may be performed. Various surgical techniques are available. Their main difference consists in filtering and non-filtering procedures. Among filtering techniques, the Gold Standard of glaucoma surgery is trabeculectomy, proposed by Cairns in the 1960s. This technique is performed by carrying out in succession, an opening of the bulbar conjunctiva (usually the upper one), with the base facing the conjunctival fornix or the sclero-corneal limbus, sculpting of a scleral flap usually of square shape, of the size of about 4×4 mm (but the flap may have other shapes, including triangular or rectangular), removal of a fragment of trabeculae, suture of the flap with usually detached sutures, and suture of the conjunctiva above. The removal of the trabeculae allows the aqueous humor to filter from the anterior chamber through the scleral flap to the subconjunctival space, forming the so-called filtering patch. The aqueous humor can then be drained by the venous stream.

An alternative procedure to trabeculectomy is the implantation of a metal device called Ex-Press®. An Ex-Press tube is positioned below the scleral flap in the thickness of the irido-sclero-corneal junction up to the anterior chamber, allowing filtration of aqueous humor in a similar way to standard trabeculectomy. The device then simulates the effect of trabeculectomy, creating an escape route from the anterior chamber to the subconjunctival space. The major difference between classic trabeculectomy and implantation of the Ex-Press device is given by the fact that the former is a manual maneuver of the surgeon who physically removes a piece of trabeculae, while the latter consists in implanting a metal tube in the trabeculae. Since the diameter of the internal lumen of the device is known (50 or 200 microns depending on the models), filtering surgery with Ex-Press is more standardized, while trabeculectomy is a manual maneuver that the surgeon performs according to his/her own skill, will, and dexterity. Even if minimally, it will always be different from one patient to another. Both surgical techniques include another manual phase depending entirely on the surgeon's responsibility, namely sculpting and subsequent closure, with sutures, of the scleral flap. Each surgeon will make a flap of different shape and size, with variability from surgeon to surgeon (interpersonal) but also from procedure to procedure (intrapersonal). The most critical phase is suturing the flap, as it determines the amount of filtration allowed by manual removal of the trabecular block (classic trabeculectomy) or through the Ex-Press device. A further alternative to trabeculectomy is the implantation of a device called InnFocus® MicroShunt:i, that is a tube with an internal diameter of 70 microns, inserted in the anterior chamber through a scleral tunnel, without creating the scleral flap. It allows outflow of aqueous humor into the subtenonal space.

Trabeculectomy presents numerous disadvantages in post-operative management, among which, possibility of hyperfiltration, with marked hypotonia of the eyeball and possibility of ex-vacuo choroid haemorrhagic detachment. Another postoperative complication is represented by the non-tightness of the conjunctival suture at the level of the filtration patch with appearance of Seidel's phenomenon. Leakage of aqueous humor through the suture of the conjunctiva also favors the hypotonia of the bulb and the possibility of postoperative infections due to direct communication between the inside and outside of the eye.

Although a lower rate of complications with Ex-press implantation has been reported in the literature, they may nevertheless occur.

The limits of the techniques described above are:

1) impossibility of regulating filtration for a single patient;

2) impossibility of standardizing surgical maneuvers;

3) unwanted filtration through the suture of the conjunctiva—with possible positive Seidel sign.

Limits 1) and 2) are attributable to the fact that the filtration level is mainly determined by greater or smaller closure of the scleral flap with the sutures entrusted to the surgeon's hands. A very tight closure of the scleral flap can cause too little or even no filtration, with hypertonicity and substantial failure of the procedure. On the contrary, a too loose suture of the scleral flap can cause hyperfiltration, with marked post-operative hypotonus and possible hemorrhagic choroid detachment. If left untreated, this complication may lead to irreversible damage to the visual function. Filtration, even in the case of InnFocus® type tube systems, cannot be modulated since the flow of aqueous humor from the anterior chamber to the outside is determined solely by the internal diameter of the device lumen.

The scleral flap may also undergo closure due to scarring. This complication is more often present in cases of too tight suture of the flap itself, with difficulty in filtration, up to its complete closure and failure of the procedure.

As for limit 3), that is filtration through the suture of the conjunctiva-positive Seidel sign, the immediate filtration that occurs as soon as the trabeculectomy or the implantation of the Ex-Press or other filtration devices such as the InnFocus® are carried out causes a sudden flow of aqueous humor towards the subconjunctival space. If the conjunctival suture is not perfectly sealed, the possibility of filtering through the suture itself may occur. An undesired and dangerous escape route for aqueous humor is therefore established as it maintains a direct connection between the external environment and the eye, with the possibility of infections up to endophthalmitis. The presence of subconjunctival fluid (aqueous humor) in the immediate postoperative period also delays the healing of the conjunctival surgical wound, especially if it is not tight, as it prevents the correct apposition of the tissues and the healing process thereof. This complication determines the need for very strict clinical controls to monitor the phenomenon and often a return to the operating room for the application of further sutures or the complete remaking of the leaky conjunctival suture.

### SUMMARY OF THE INVENTION

Therefore, a need is felt to overcome the drawbacks and limitations mentioned above with reference to the prior art.

In particular, the need is felt to overcome the following draw-backs/limitations of current filtering surgery techniques for open-angle glaucoma:

1) non-modulable/adjustable/controlled/customizable filtration for the individual patient;

2) filtration through the conjunctiva towards the outside of the eye in the immediate postoperative period with the onset of the so-called positive Seidel sign, due to filtration of the aqueous humor through the conjunctival wound not yet sealed;

3) as an effect of a solution of the problem referred to in point 2 (abnormal and undesired filtration through the conjunctival suture) is the resolution in many cases of the postoperative complication of the ocular hypotonus and the consequent onset of possible haemorrhagic detachment of ex vacuo choroid.

Such a need is met by a device as described and claimed herein.

### BRIEF DESCRIPTION OF THE DRAWINGS

Further features and advantages of the present invention will be more comprehensible from the following description of preferred embodiments given by way of non-limiting examples, in which:

FIG. 1 shows a perspective view of a medical device for glaucoma surgery according to an embodiment of the present invention;

FIG. 2 shows a lateral view of the device of FIG. 1 from the side of arrow II indicated in FIG. 1;

FIG. 3 is a lateral view of the medical device of FIG. 1 from the side of arrow III indicated in FIG. 1;

FIG. 4 is a lateral view of the medical device of FIG. 1 from the side of arrow IV indicated in FIG. 1;

FIGS. 5-6 show a perspective view and a lateral view, respectively, of a medical device for glaucoma surgery according to a further embodiment of the present invention;

FIG. 7 shows a front perspective view of a medical device for glaucoma surgery according to a further embodiment of the present invention;

FIG. 8 shows a perspective view of the front detail VIII of the medical device for glaucoma surgery of FIG. 7;

FIG. 9 shows a rear perspective view of the medical device for glaucoma surgery of FIG. 7;

FIG. 10 shows a top view of the medical device for glaucoma surgery of FIG. 7;

FIG. 11 shows a lateral view of the medical device for glaucoma surgery of FIG. 7; and

FIGS. 12a-12b show sectional views of a medical device for glaucoma surgery of the prior art and of a medical device for glaucoma surgery according to the present invention applied to a sclera, respectively.

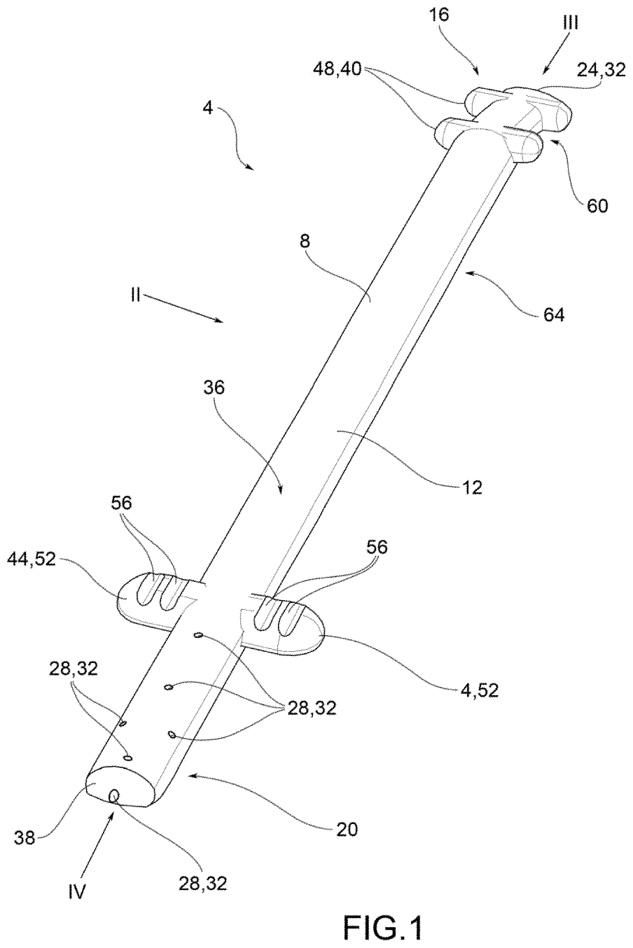

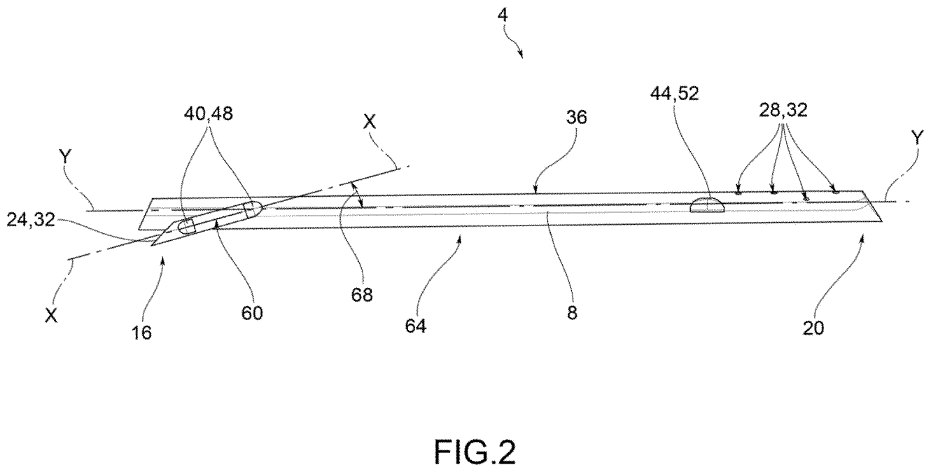

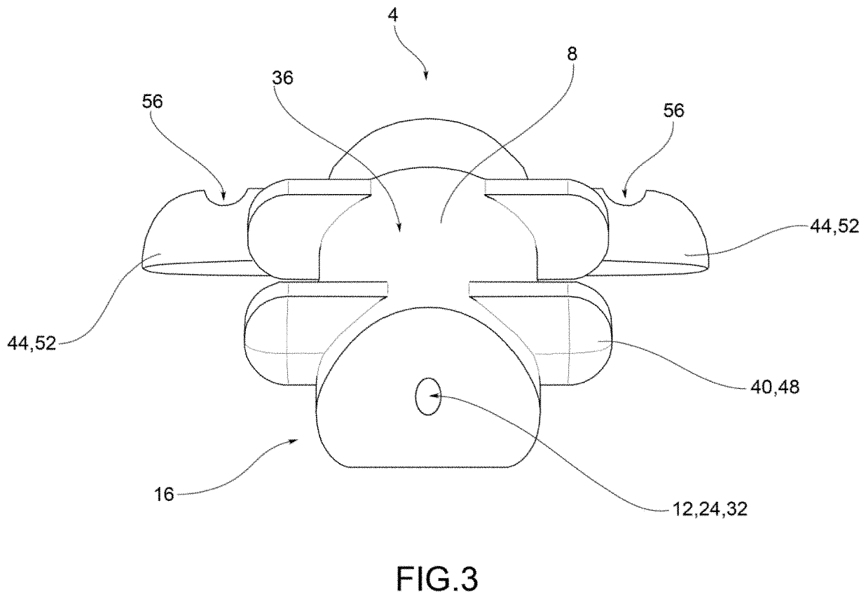

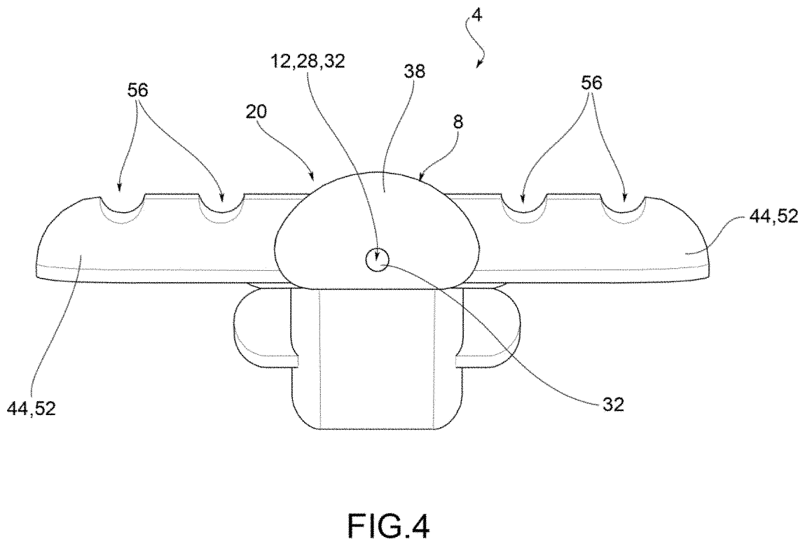

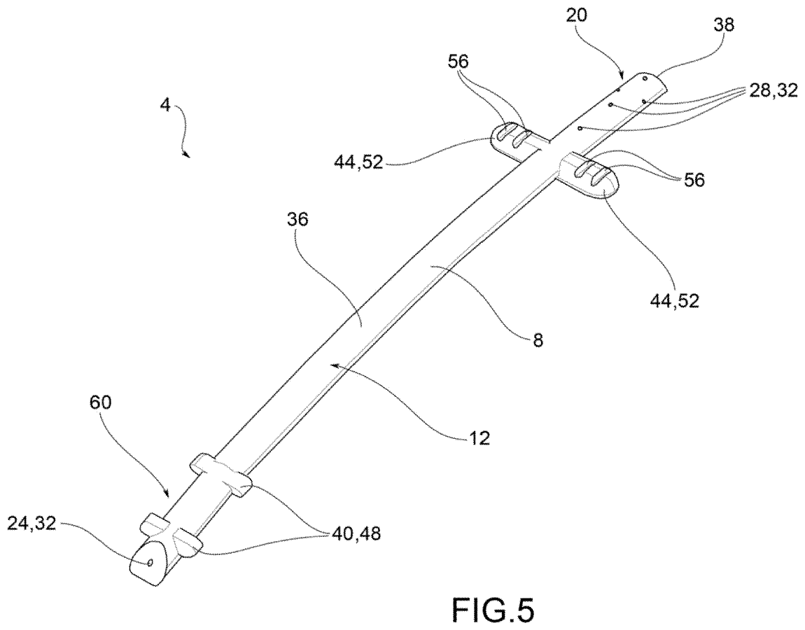

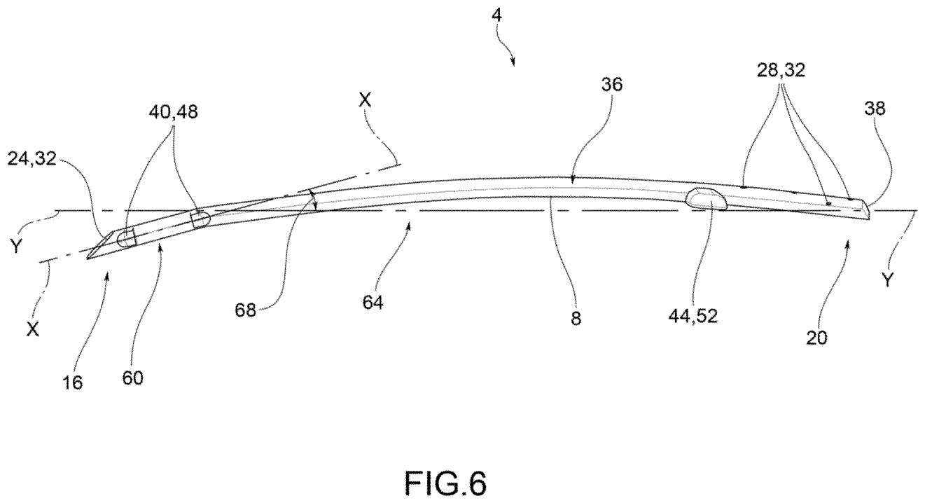

Elements or parts in common to the embodiments described will be indicated hereafter using the same reference numerals.

### DETAILED DESCRIPTION

With reference to the aforementioned figures, reference numeral 4 globally indicates a medical device for glaucoma surgery.

The medical device 4 comprises a tubular body 8 provided with a through cavity 12 extending from a proximal or intraocular end 16 to a distal or extraocular end 20. The tubular body 8 may be made of plastic, polymeric, metal, hypoallergenic, biocompatible material and the like.

The tubular body 8 comprises at least one proximal filtration hole 24, located on the side of the proximal end 16, and at least one distal filtration hole 28, located on the side of the distal end 20.

Said proximal and distal filtration holes 24, 28 are fluidically connected to each other by the through cavity 12. In other words, the through cavity 12 puts the at least one proximal filtration hole 24, located on the side of the proximal end 16, and the at least one distal filtration hole 28, located on the side of the distal end 20, in fluid communication with each other.

Preferably, the at least one proximal filtration hole 24 is located at the proximal end 16, and the at least one distal filtration hole 28 is located on an upper and upper-lateral surface 36 of the tubular body 8. The at least one distal filtration hole 28 may be located on a rear surface 38 of the tubular body 8.

By at least one hole it is meant that it is possible to provide a single filtration hole 24, 28 or also a plurality of filtration holes 24, 28. The plurality of filtration holes may comprise a set of holes arranged according to geometric patterns adapted to promote an adequate dispersion of the drained liquid through the through cavity 12.

According to a possible embodiment, the through cavity 12 has a passage width or lumen having an internal diameter ranging between 40 microns and 140 microns.

Preferably, the tubular body 8, with respect to a section plane perpendicular to a prevailing direction of extension of the medical device 4 which connects the proximal or intraocular end 16 with the distal or extraocular end 20, has an at least partially concave section 22 at a side or lower face 23 suitable for interfacing with an ocular sclera 25. FIGS. 12a and 12b illustrate the interaction between the tubular body 8 and the sclera 25 in a solution of the prior art (FIG. 12a) and in a solution according to the present invention (FIG. 12b). In the solution of the prior art (FIG. 12a), the tubular body 8 has a circular section and, therefore, it imprints the underlying sclera 25 in an ideally point-like manner. In this way, the contact pressure is relatively high. On the other hand, in the solution according to the present invention (FIG. 12b), the tubular body 8 has an at least partially concave section 22 at a side or lower face 23. In this way, the tubular body 8 may rest on the sclera 25 in a more uniform manner due to an overall wider support surface, so as to distribute the contact pressure over a wider area of sclera 25.

According to an embodiment of the present invention, the tubular body 8, with respect to a section plane perpendicular to a prevailing direction of extension of the medical device 4 which connects the proximal or intraocular end 16 to the distal or extraocular end 20, has a convex, elliptical or parabolic section, at a side or upper face 27, suitable for interfacing with the conjunctiva 29. Also in this case, with respect to the point-like contact area of the circular solutions of the prior art (FIG. 12a), the present invention (FIG. 12b) allows obtaining a more rounded or less steep course of the conjunctiva 29 above and a more uniform contact pressure between the conjunctiva 29 and the upper face 27 of the tubular body 8. A more rounded soft course means a wider curvature that allows increasing the surfaces in mutual contact and, therefore, decreasing the specific contact pressure between them.

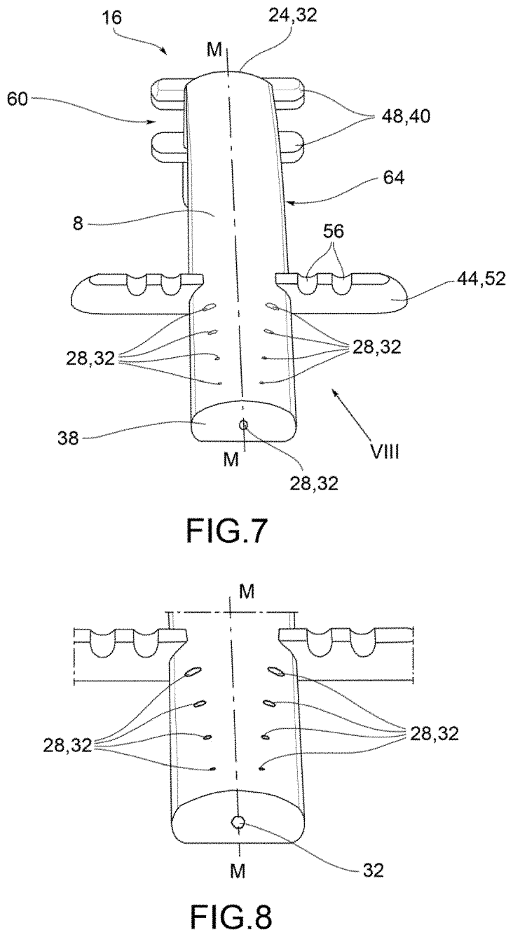

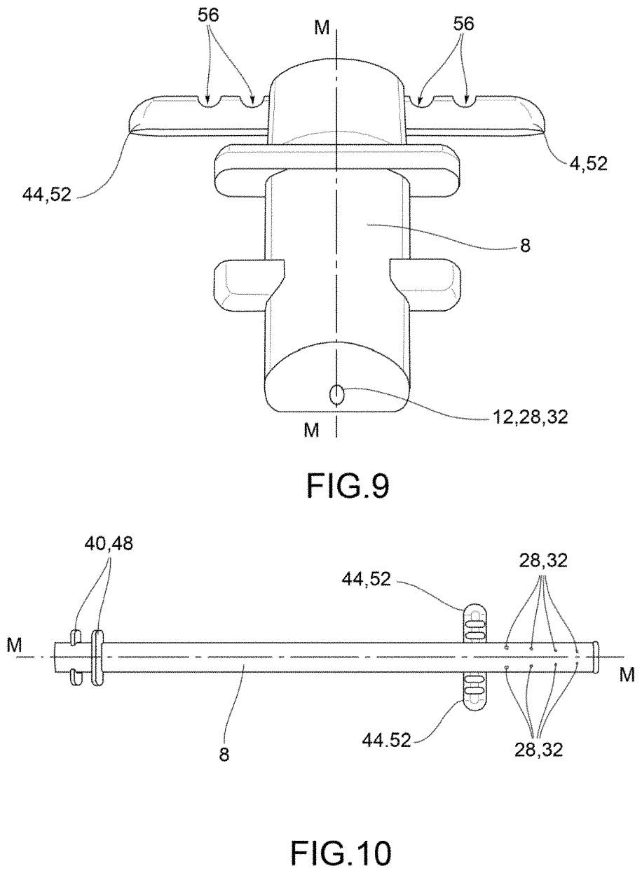

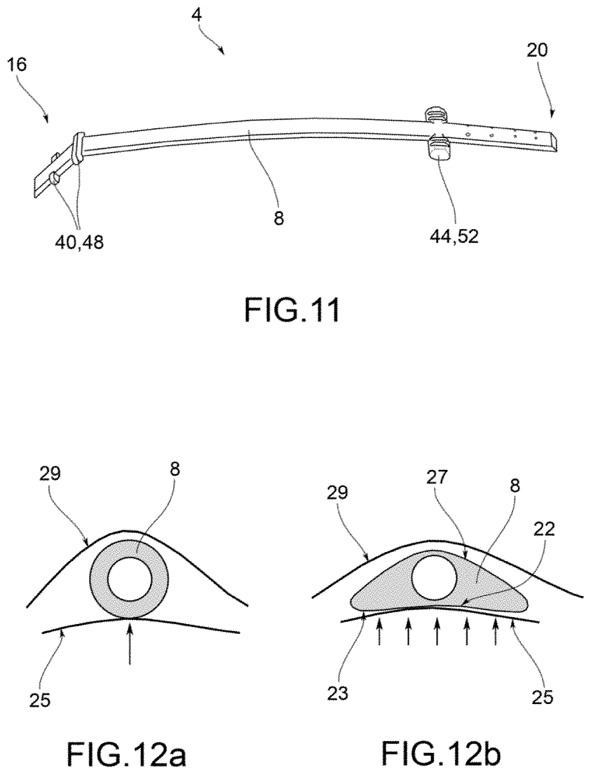

According to a possible embodiment (FIGS. 7-11), the tubular body 8 comprises a plurality of distal filtration holes 28 having different diameters. In particular, said distal filtration holes 28 have an increasing diameter moving from the distal end 20 towards the proximal end 16. Said distal filtration holes 28 with variable diameter allow choosing the quantity of filtration flow based on the pressure of the individual patient. They also allow a progression of the amount of flow, as the ophthalmologist can begin to open the distal filtration hole 28 with a smaller diameter and then subsequently act on, for example, one or two holes of greater diameter if the intraocular pressure conditions require it.

Furthermore, according to a possible embodiment (FIGS. 7-11), said distal filtration holes 28 are at least partially located on opposite sides with respect to a median plane M-M of the tubular body 8 so as to allow, by means of differentiated openings, having a flow directed more to the left or to the right of said median plane M-M of the tubular body 8.

Advantageously, said proximal and distal filtration holes 24, 28 are at least partially or completely occluded by a membrane 32 which can be at least partially removed by selective action, so as to allow selective variation of the amount of aqueous humor flow removable via said through cavity 12.

According to a possible embodiment, said membrane 32 is a membrane that can be perforated or removed by means of a stylet.

According to a possible embodiment, said membrane 32 is a membrane that can be perforated or removed by application of a laser source from the outside.

According to a further possible embodiment, said membrane 32 is a mobile membrane allowing patency of the corresponding at least one proximal or distal occluding filtration hole 24, 28 to be varied.

According to a further possible embodiment, said membrane 32 is a resorbable membrane in contact with aqueous humor. The reabsorption of the membrane can be established or predetermined in a predetermined time, not being instantaneous. This predetermined time can be varied at will by acting, for example, on the size and/or thickness and/or material of the reabsorbable membrane 32.

It should be noted that the embodiments of membrane 32 described above are not necessarily alternative to each other; in other words, they may coexist in the same embodiment.

For example, the membrane 32 can be perforated and removed by a stylet and/or by a laser source. It can also be mobile and/or resorbable in contact with aqueous humor in a time predetermined by the features of the resorbable material and/or by the geometry of the membrane.

All the aforementioned embodiments constitute alternative or synergistic variants which allow exactly dosing or calibrating the overall patency of the medical device 4 and, therefore, the aqueous humor drainage/filtering capacity.

According to an embodiment, at the proximal end 16, the tubular body 8 comprises a first proximal positioning means 40 of the medical device 4, while, on the side of the distal end 20, the tubular body 8 comprises a second distal positioning means 44 of the medical device 4.

According to an embodiment, the first proximal positioning means 40 of the tubular body 8 comprises a pair of rings or positioning protuberances 48.

According to an embodiment, said rings or positioning protuberances 48 are arranged in positions diametrically opposite to the tubular body 8 itself.

For example, said rings or positioning protuberances 48 are axially spaced from each other by a distance of between 0.5 and 3 mm, so as to position themselves astride the sclera-cornea junction. This conformation allows the medical device 4 to remain in place in the irido-sclero-corneal junction, preventing it from being displaced intraocularly or being extruded out of the eye.

According to an embodiment, the second distal positioning means 44 comprises a pair of positioning and attachment tabs 52 for positioning and attaching to the ocular sclera. These positioning and attachment tabs 52 allow the medical device 4 to remain in place, preventing it from moving on the sclera and causing a dislocation thereof with respect to the design working position at the level of the irido-sclero-corneal junction.

Preferably, said positioning and attachment tabs 52 are arranged in positions diametrically opposite to the tubular body 8.

According to a possible embodiment, said positioning and attachment tabs 52 are provided with seats 56 for receiving suture threads.

According to a further possible embodiment, said positioning and attachment tabs 52 are smooth, that is, without said seats 56, and are shaped in such a way as to be able to be inserted in scleral pockets without sutures.

Preferably, the rings or positioning protuberances 48 and the positioning and attachment tabs 52 are angularly aligned with each other with respect to the tubular body 8.

According to an embodiment, the tubular body 8 comprises a first proximal tract 60, provided with the first proximal positioning means 40, and a second distal tract 64, provided with the second distal positioning means 44.

According to a possible embodiment, said first proximal tract 60 and second distal tract 64 are rectilinear, respectively, along a first prevailing direction X-X and a second prevailing direction Y-Y, preferably inclined with each other by an angle of deflection 68 between 25 and 45 degrees. In this way, the tubular body 8 is able to position itself radially along the sclera.

According to a further embodiment, the profile of said second distal tract 64 is slightly curved according to the radius of curvature of the sclera in the prevailing direction Y-Y.

For example, the first proximal tract 60 has an extension along the first prevailing direction X-X between 1.5 and 3.5 mm, while the second distal tract 64 has an extension along the second prevailing direction Y-Y between 6 and 9 mm.

The method of use and the operation of the medical device according to the present invention will now be described.

In particular, the device is positioned on the side of its proximal end 16 at the irido-sclero-corneal junction of the patient.

The flow of aqueous humor, exiting the eye through the medical device 4, begins when the reabsorption of the reabsorbable membrane/cap 32 occurs at the level of the endocular hole or the proximal filtration hole 24 (or it is opened by a YAG laser) and/or when the reabsorption of the reabsorbable membrane/cap 32 occurs at the level of the distal filtration hole 28, or the opening of at least one of the distal filtration holes 28 is made on the upper or upper-lateral surface 36 of the medical device 4 itself by the methods described above.

The opening of the filtration holes 24, 28 is made directly or occurs by reabsorption of the membrane 32 after a suitable number of days, so as to give the conjunctival wound time to heal and be sealed. The amount or volume of filtration is determined by the diameter and number of openings of the filtration holes 24, 28 on the extraocular segment.

As mentioned above, opening of the filtration holes 24, 28 may take place in various ways.

Opening of the filtration holes 24, 28 may take place by pressure with a metal stylet (or alternatively also of other materials) thereon so as to obtain yielding thereof towards the inside of the tubular body 8 according to a programmed, controlled and predetermined process. Each opening of a filtration hole 24, 28 causes a release of a predetermined quantity of microliters of aqueous humor/hour. Filtration holes 24, 28 may be opened also by treatment with an argon-type thermal laser or by a YAG laser. The material of the holes may be the same as the structure of the medical device 4 or different, according to the needs and opening methods.

The proximal hole 24 of the endocular ending is preferably closed by a membrane of resorbable material and therefore is not patent at the time of implantation. The closure material has the feature of being resorbable in about 1-15 days. Alternatively, the closure may be made with non-resorbable material but sensitive to treatment with a YAG laser which causes the destruction of the material itself and the consequent patency of the medical device 4.

Therefore, the implant does not determine any filtration in the immediate postoperative period (as in known solutions). This specific feature allows the surgery-induced conjunctival wound to heal before the flow of aqueous humor begins.

As can be seen from the above description, the medical device according to the present invention allows the drawbacks of the prior art to be overcome.

In particular, the present invention allows modulating the filtration in the postoperative period in a selective, progressive and controlled manner according to the intraocular pressure of the single patient.

Furthermore, the device allows solving the problem of abnormal filtration through the conjunctival suture in the immediate postoperative period.

Furthermore, the device allows solving the problem of abnormal filtration due to the fact that filtration is not immediate, but occurs only after the conjunctiva has healed correctly.

As described above, the suture of the scleral flap is made to be sealed. The aqueous humor does not filter through the scleral flap (as in the solutions of the prior art) but filters through the filtration holes outside the same. The unpredictability caused by surgeon's hands in placing the sutures of the flap is therefore eliminated. Therefore, in this case the scleral flap has only the protective function of the implanted device and no longer determines the quantity or volume of the aqueous humor flow (as occurs in the solutions of the prior art).

Furthermore, as seen, the positioning tabs allow both the suture of the implant to the sclera and their positioning in scleral pockets without the need for sutures.

A person skilled in the art may make several changes and adjustments to the medical devices described above in order to meet specific and incidental needs, all falling within the scope of protection .as described and claimed herein.

What is claimed is:

1. A medical device for glaucoma surgery, the medical device comprising:
a tubular body comprising a through cavity extending from a proximal or intraocular end to a distal or extraocular end, wherein:
at the proximal or intraocular end, the tubular body comprises a first proximal positioning means of the medical device,
at the distal or extraocular end, the tubular body comprises a second distal positioning means of the medical device,
the tubular body comprises at least one proximal filtration hole, located on a side of the proximal or intraocular end, and at least one distal filtration hole, located on the side of the distal or extraocular end, said at least one proximal and distal filtration holes, being fluidically connected to each other by said through cavity, and
said at least one proximal and distal filtration holes are at least partially or completely occluded by a membrane at least partially removable by selective action, so as to allow a selective variation of an amount of aqueous humor flow removable via said through cavity.

2. The medical device of claim 1, wherein said membrane is perforable or removable by a stylet and/or by application of a laser source.

3. The medical device of claim 1, wherein said membrane is a mobile membrane allowing patency of at least one corresponding proximal or distal filtration hole to be varied.

4. The medical device of claim 1, wherein said membrane is resorbable in contact with aqueous humor in a predetermined time range.

5. The medical device of claim 1, wherein the through cavity has a through width or lumen with an internal diameter ranging between 40 and 140 microns.

6. The medical device of claim 1, wherein the at least one proximal filtration hole is located at the proximal or intraocular end and wherein the at least one distal filtration hole is located on an upper and/or upper-rear and/or upper-lateral surface of the tubular body.

7. The medical device of claim 1, wherein the tubular body comprises a plurality of distal filtration holes having increasing diameter moving from the distal or extraocular end towards the proximal or intraocular end.

8. The medical device of claim 1, wherein the tubular body comprises a plurality of distal filtration holes at least partially located on opposite sides with respect to a median plane (M-M) of the tubular body.

9. The medical device of claim 1, wherein the tubular body, with respect to a section plane perpendicular to a prevailing direction of extension of the medical device connecting the proximal or intraocular end to the distal or extraocular end, has an at least partially concave section at a side or lower face suitable for interfacing with an ocular sclera.

10. The medical device of claim 1, wherein the tubular body, with respect to a section plane perpendicular to a prevailing direction of extension of the medical device connecting the proximal or intraocular end to the distal or extraocular end, has a convex, elliptical or parabolic section, at a side or upper face suitable for interfacing with a conjunctiva.

11. The medical device of claim 1, wherein the first proximal positioning means of the tubular body comprises a pair of rings or positioning protuberances arranged in positions diametrically opposite to the tubular body, and wherein said pair of rings or positioning protuberances are axially spaced from each other by a distance of between 0.5 mm and 3 mm, so as to position themselves astride the sclera-cornea junction.

12. The medical device of claim 1, wherein the second distal positioning means comprises a pair of positioning and attachment tabs for positioning and attaching to the ocular sclera, wherein said positioning and attachment tabs are arranged in positions diametrically opposite to the tubular body.

13. The medical device of claim 12, wherein said positioning and attachment tabs are provided with seats for receiving suture threads.

14. The medical device of claim 12, wherein said positioning and attachment tabs are smooth and shaped so as to be inserted into scleral pockets without sutures.

15. The medical device of claim 11, wherein the second distal positioning means comprises a pair of positioning and attachment tabs and wherein the positioning rings or protuberances and the positioning and attachment tabs are aligned angularly with each other with respect to the tubular body.

16. The medical device of claim 1, wherein the tubular body comprises a first proximal tract, provided with the first proximal positioning means, and a second distal tract, provided with the second distal positioning means, said first proximal and second distal tracts being rectilinear, respectively, along a first and second prevailing directions (X-X, Y-Y), said first and second prevailing directions (X-X, Y-Y) being inclined with each other by an angle of deflection ranging between 25 and 45 degrees.

17. The medical device of claim 1, wherein the tubular body comprises a first proximal tract, provided with the first proximal positioning means, and a second distal tract, provided with the second distal positioning means, said first proximal and second distal tracts being directed respectively along a first and a second prevailing directions (X-X, Y-Y), wherein said second distal tract is slightly curved according to curvature radius of the sclera in the second prevailing direction.
